(19) United States
(12) Patent Application Publication
(10) Pub. No.: US 2025/0249183 A1
(43) Pub. Date: Aug. 7, 2025

(54) DENTAL GUIDE

(71) Applicant: Eric David Turner, Jr., Charlotte, NC (US)

(72) Inventor: Eric David Turner, Jr., Charlotte, NC (US)

(21) Appl. No.: 18/816,187

(22) Filed: Aug. 27, 2024

Related U.S. Application Data
(60) Provisional application No. 63/550,874, filed on Feb. 7, 2024.

(51) Int. Cl. A61M 5/42

(52) U.S. Cl. CPC A61M 5/427; A61M 2207/00; A61M 2210/0631

(57) ABSTRACT

Apparatuses, methods of use, and methods of manufacture are described herein for a dental guide. The dental guide may include an elongate member defining a first end and a second end, a needle support proximate the first end, the needle support defining an aperture structured to receive a needle, and a fixed hub proximate the second end.

### CROSS-REFERENCE TO RELATED APPLICATIONS

This application claims the benefit of U.S. Provisional Patent Application Ser. No. 63/550,874, filed Feb. 7, 2024, titled DENTAL GUIDE, the content of which is hereby incorporated by reference in its entirety.

### FIELD

The present disclosure relates to a dental guide and methods of generation and use of a dental guide.

### BACKGROUND

Current dental anesthesia administration methods face challenges due to the variability in nerve and foramen locations. The reliability of oral landmarks to guide injection placement and technique is limited, and the common approach involves puncturing the skin, penetrating until the jawbone is reached, blindly repositioning the needle, and covering a wide area with anesthetic in hopes that the nerve lies along the chosen needle path.

Applicant has identified a number of deficiencies and problems associated with the commonly accepted technique for dental anesthesia, including issues with the architecture of the jawbone, the soft tissues of the mouth, and the diffusion of numbing medicine. These deficiencies often necessitate the need for multiple injections, leading to tissue and nerve damage. Many of these identified problems have been solved by developing solutions that are included in implementations of the present disclosure, many examples of which are described in detail herein.

### SUMMARY

The following presents a simplified summary of one or more implementations of the present disclosure, in order to provide a basic understanding of such implementations. This summary is not an extensive overview of all contemplated implementations and is intended to neither identify key or critical elements of all implementations nor delineate the scope of any or all implementations. Its sole purpose is to present some concepts of one or more implementations of the present disclosure in a simplified form as a prelude to the more detailed description that is presented later.

In one aspect, a dental guide is presented. The dental guide may include an elongate member defining a first end and a second end, a needle support proximate the first end, the needle support defining an aperture structured to receive a needle, and a fixed hub proximate the second end.

In some implementations, the fixed hub may include a contoured portion structured to receive a portion of at least one tooth.

In some implementations, the fixed hub may include a first material and the contoured portion may include a second material.

In some implementations, the aperture may include a bushing having an inner diameter sufficient to receive the needle.

In some implementations, the bushing may include a metal.

In some implementations, at least one of the elongate member, the needle support, and the fixed hub, may include an additive manufacturing material.

In some implementations, the additive manufacturing material may include a polymer.

In some implementations, additive manufacturing material may include a metal.

In some implementations, the elongate member defines a longitudinal axis, and the needle support extends from the elongate member in a direction substantially orthogonal to the longitudinal axis of the elongate member.

In some implementations, the needle support defines a lingual surface for receiving a proximal end of the needle.

In some implementations, when installed on a patient having a mandibular foramen, the lingual surface is positioned between approximately 6 mm and approximately 50 mm from the mandibular foramen.

In another aspect, a method for injecting an anesthetic is presented. The method may include applying a dental guide to at least one tooth of a patient, and the dental guide may include an aperture for providing access to a mandibular foramen, receiving an injection device in the aperture, and the injection device is structured to deliver anesthetic, advancing the injection device through the aperture until a delivery tip of the injection device is positioned proximate the mandibular foramen, and injecting the anesthetic upon the delivery tip being proximate the mandibular foramen.

In some implementations, the method may further include removing the injection device through the aperture of the dental guide and removing the dental guide from the at least one tooth of the patient.

In some implementations, the injection device may include a needle of a syringe.

In some implementations, the dental guide may include a fixed hub having a contoured portion and applying the dental guide to the at least one tooth of the patient may include applying a temporary adhesive between the contoured portion and the at least one tooth.

In yet another aspect, a method of manufacturing a dental guide is presented. The method may include receiving image data of a patient, the image data having a mandibular foramen of the patient, applying a linear marker in the image data to a position proximate the mandibular foramen, determining an orientation of the linear marker based on anatomical features of the patient represented in the image data, forming a needle support based on the orientation of the linear marker, forming a fixed hub based on the anatomical features of the patient represented in the image data, and forming an elongate member extending between the needle support and the fixed hub.

In some implementations, the image data may include first image data from an optical scan of the patient and second image data from a Computed Tomography (CT) scan of the patient.

In some implementations, the method may further include arranging first image data of the patient and second image data of the patient to form combined image data, and the image data of the patient may include the combined image data.

In some implementations, at least one of forming the needle support, forming the fixed hub, and forming the elongate member may include using additive manufacturing.

In some implementations, forming the needle support may include forming an aperture in the needle support for receiving a needle.

The above summary is provided merely for purposes of summarizing some example implementations to provide a basic understanding of some aspects of the present disclosure. Accordingly, it will be appreciated that the above-described implementations are merely examples and should not be construed to narrow the scope or spirit of the disclosure in any way. It will be appreciated that the scope of the present disclosure encompasses many potential implementations in addition to those here summarized, some of which will be further described below.

### BRIEF DESCRIPTION OF THE DRAWINGS

Having thus described implementations of the disclosure in general terms, reference will now be made to the accompanying drawings, wherein:

FIG. 1A illustrates a top plan view of a mandible represented by image data, in accordance with an implementation of the disclosure;

FIG. 1B illustrates a perspective view of a mandible represented by image data, in accordance with an implementation of the disclosure;

FIG. 2A illustrates a front view of a mandible represented by image data and a corresponding dental guide, in accordance with an implementation of the disclosure;

FIG. 2B illustrates a top plan view of a mandible represented by image data and a corresponding dental guide, in accordance with an implementation of the disclosure;

FIG. 3A illustrates a perspective view of a pair of dental guides, in accordance with an implementation of the disclosure;

FIG. 3B illustrates a bottom plan view of a pair of dental guides, in accordance with an implementation of the disclosure;

FIG. 4A illustrates a perspective view of a dental guide, in accordance with an implementation of the disclosure;

FIG. 4B illustrates a left side view of a dental guide, in accordance with an implementation of the disclosure;

FIG. 4C illustrates a right side view of a dental guide, in accordance with an implementation of the disclosure;

FIGS. 5A-5C illustrate technical components of an exemplary distributed computing environment for generation of dental guides, in accordance with an implementation of the disclosure;

FIG. 6 illustrates an exemplary machine learning (ML) subsystem architecture 600, in accordance with an implementation of the disclosure;

FIG. 7 illustrates a process flow of the generation of a dental guide, in accordance with an implementation of the disclosure; and

FIG. 8 illustrates a process flow of the administration of an anesthetic using a dental guide, in accordance with an implementation of the disclosure.

### DETAILED DESCRIPTION

Implementations of the present disclosure now may be described more fully hereinafter with reference to the accompanying drawings, in which some, but not all, implementations of the disclosure are shown. Indeed, the disclosure may be embodied in many different forms and should not be construed as limited to the implementations set forth herein; rather, these implementations are provided so that this disclosure may satisfy applicable legal requirements. Like numbers refer to like elements throughout. Where possible, any terms expressed in the singular form herein are meant to also include the plural form and vice versa, unless explicitly stated otherwise. Also, as used herein, the term “a” and/or “an” shall mean “one or more,” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on.”

Additionally, certain terminology is used herein for convenience only and is not to be interpreted as a limitation on the implementations described. For example, the words “top,” “bottom,” “upper,” “lower,” “left,” “right,” “horizontal,” “vertical,” “upward,” and “downward” merely describe the configurations as depicted in the figures. Indeed, the referenced components in the figures may be oriented in any direction, unless specified otherwise, the configurative terminology used herein should be understood as encompassing such variations.

It should also be understood that “operable communication” or “operably coupled” as used herein, means that the components may be formed integrally with each other, or may be formed separately and coupled together. Furthermore, “operable communication” means that the components may be formed directly to each other, or to each other with one or more components located between the components that are operatively coupled together. Furthermore, “operable communication” or “operably coupled” may mean that the components are detachable from each other, or that they are permanently coupled together. Furthermore, components in operable communication may mean that the components retain at least some freedom of movement in one or more directions or may be rotated about an axis (i.e., rotationally coupled, pivotally coupled). Furthermore, “operable communication” or “operably coupled” may mean that components may be electronically connected and/or in fluid communication with one another.

In the context of dental procedures, bone density plays a pivotal role in the effectiveness of anesthetizing techniques. The diffusion of anesthetic agents (including infiltration anesthetics) is significantly influenced by the thickness of the bone surrounding the teeth. Targeted injections such as nerve block(s) may be implemented, which anesthetize larger areas (e.g., multiple teeth and larger tissue areas) and prevent the need for multiple injections, or to bolster the effectiveness of local anesthesia in more complex dental procedures. In cases involving the upper teeth and the lower front teeth, the relatively thin veneer of bone facilitates straightforward numbing, allowing infiltration anesthetics to be deposited directly under the skin near the tooth and permeate effectively to cause tooth anesthesia. However, this scenario contrasts markedly with the lower back teeth. Here, the bone density is substantially higher, presenting a formidable barrier for the diffusion of conventional anesthetic agents. Consequently, depositing anesthetic in close proximity to these teeth, external to their dense bony housing, poses a significant technical challenge in dentistry.

One approach to numb lower back teeth involves targeting the inferior alveolar nerve at its entry into the mandibular foramen, which is nestled within the bony housing of the jawbone and shielded by a projection of bone known as the lingula. The current method, known as an inferior alveolar nerve block, relies on a technique that is more art than science. The approach most commonly used for anesthesia of the inferior alveolar nerve in the United States is the traditional Halstead method, a technique in which the inferior alveolar nerve is reached by an intraoral access before it penetrates the mandibular canal. Dentists typically use a “spray and pray” approach with a needle, penetrating the inside of the cheek perpendicular to the jaw bone, hitting the bone, pivoting the needle, and then blindly penetrating the deeper cheek tissues (e.g., muscles, ligaments, tendons, blood vessels) such that the needle travels parallel to the medial wall of the ramus of the mandible in an attempt to reach the nerve. The anatomic variability in the location of the nerve relative to visible landmarks in the mouth complicates the process, making it challenging to accurately deposit anesthetic close to the nerve. Depositing anesthetic too far from the nerve often results in inadequate anesthesia due to the limited ability of the anesthetic to diffuse through the nearby soft tissues. Furthermore, the Halstead technique often results in the needle being obstructed by the lingula and/or by a curving out ridged architecture of the mandible as the lingula and foramen are approached by the needle.

Thus, the traditional injection technique often requires multiple injections with the same needle, leading to tissue damage, soreness, and the potential for nerve damage. The imprecise deposition of anesthesia, combined with the need for multiple injections, contributes to an increased likelihood of adverse effects, such as long-term numbness, tingling, burning sensations, and paresthesia. The current injection technique is described as inaccurate, contributing to increased tissue damage and soreness, highlighting the need for a more precise and reliable solution to address the challenges associated with anesthetic and nerve block implementation in dentistry.

Implementations of the disclosure described herein address these challenges by embracing a dental guide that allows for the introduction of an anesthetic to a target area proximate the inferior alveolar nerve by targeting the mandibular foramen, neck of the mandibular condyle, or the like. Traditional methods may rely on “soft” tissue landmarks that change shape and position inside the mouth as a result of changes to soft tissue or changes in the position of the mandible and/or neck during a procedure. Importantly, the dental guide presented herein leverages the constant relative positioning between the teeth of a patient and the mandibular foramen, mandibular condyle, or the like, which are “hard” bone landmarks.

It shall be appreciated that although reference is made in the present disclosure to a “needle tip” or “tip of a needle”, a needle tip is only one example of a “delivery tip”, as other anesthetic delivery methods may be implemented. Indeed, the “delivery tip” refers to the point from which anesthetic is released from various types of anesthetic delivery devices (i.e., injection devices), including from the tip of a needle of needle-based injection devices, delivery tips of needle-free injection devices that deliver anesthetic through tissue using high pressures, or the like. Similarly, while the present disclosure may reference a “needle” in relation to various dental guide sizing parameters or anesthetic application methods, the term “needle” merely refers to a passageway or conduit of anesthetic that transports anesthetic to the targeted area and is not limited to traditional needle-based injection devices. Thus, the “needle” serves to orient the delivery tip regardless of the anesthetic delivery device used.

While references may be made throughout the present disclosure to the mandibular foramen and the placement of the delivery tip of the injection device (and as a result, the anesthetic) proximate the mandibular foramen, it shall be appreciated that the techniques and implementations described herein may be similarly applied to any other anatomical landmark suitable for the application of anesthetic and accessible through the mouth of a patient. This includes, but is not limited to, applying the target area at the neck of the mandibular condyle, another known location of the inferior alveolar nerve, for completion of a “Gow Gates” nerve block. Furthermore, the concept and devices for guiding anesthesia described herein may also be applied to the intraosseous injection technique, during which a delivery tip delivers anesthetic to the spongy bone surrounding the roots of the teeth, where precise guidance is critical to avoid damaging the roots of the teeth during the injection process.

Accordingly, as used herein, an “anatomical landmark” may refer to the bone structure that provides a means of access to nerves within or surrounding the mandible, including, but not limited to, the inferior alveolar nerve. For example, an “anatomical landmark” may refer to the mandibular foramen, mandibular condyle, or the like.

Similarly, as used herein, a “target area” may refer to a position or placement of the delivery tip of a needle relative to the anatomical landmark for the administration of anesthetic. For implementations directed to the mandibular foramen as the anatomical landmark, the “target area” may refer to one or more locations proximate an upper portion of the mandibular foramen. For implementations directed to the mandibular condyle as the anatomical landmark, the “target area” may refer to the “neck” of the mandibular condyle (i.e., the relatively narrow portion of the mandibular condyle that connects the head of the condyle to the shaft of the mandible). Indeed, it will be appreciated that additional target areas may be defined as needed for optimal administration of anesthetic and/or any other anatomical landmarks.

The dental guide may be generated electronically using image data from various scans of a human mandible, including Computed Tomography (“CT”) scan data and other image data to represent the anatomical features of a patient electronically on an interface. Based on the image data, a linear marker is placed at a target area, such location being determined either visually by a user or via a machine learning model trained to isolate the anatomical landmark(s) and its respective target area(s) from the remainder of the anatomy based on training data provided to the model. Similarly, the orientation of the linear marker (e.g., the angle of the linear marker relative to the x, y, and z axes) may be determined visually, based on the operator's familiarity with the soft tissue and other anatomy of the mandible. Alternatively, the orientation may be determined through a machine learning model, the machine learning model having been trained on sample datasets containing the most probable locations of the soft tissue based on the bone structure of the mandible.

Needle support rendering data to form a needle support is generated to surround the linear marker, with the linear marker serving to represent an aperture through which the needle will be inserted during use of the dental guide. Accordingly, the longitudinal axis of the aperture within the needle support rendering data will be collinear to that of the longitudinal axis of the linear marker, such that any needle inserted through the aperture will be directed towards the target area. The position of the proximate surface of the needle support rendering data (i.e., the lingual surface) will be generated at a predetermined distance equal to that of a length of a needle. Accordingly, the delivery tip through which the anesthesia is applied will be the optimal distance from the anatomical landmark in a manner repeatable each time a needle is fully inserted through the aperture. Next, image data specific to teeth are used to form the fixed hub rendering data, which pertains to a three-dimensional structure for surrounding at least one tooth. The anatomical data of the teeth may be scaled or translated in one or more directions to facilitate the insertion and removal of the fixed hub, which is fabricated based on the rendering data formed for the fixed hub rendering data, to be inserted and removed from the at least one tooth. The fixed hub rendering data (e.g., the 3D model of the fixed hub) and the needle support rendering data (e.g., the 3D model of the needle support) are then connected by elongate member rendering data (e.g., a 3D model of the elongate member), which provides support to the needle support and positions the needle support relative to the fixed hub. Using the combined rendering data from at least one of the fixed hub rendering data, the needle support rendering data, and the elongate member rendering data, the dental guide may be fabricated.

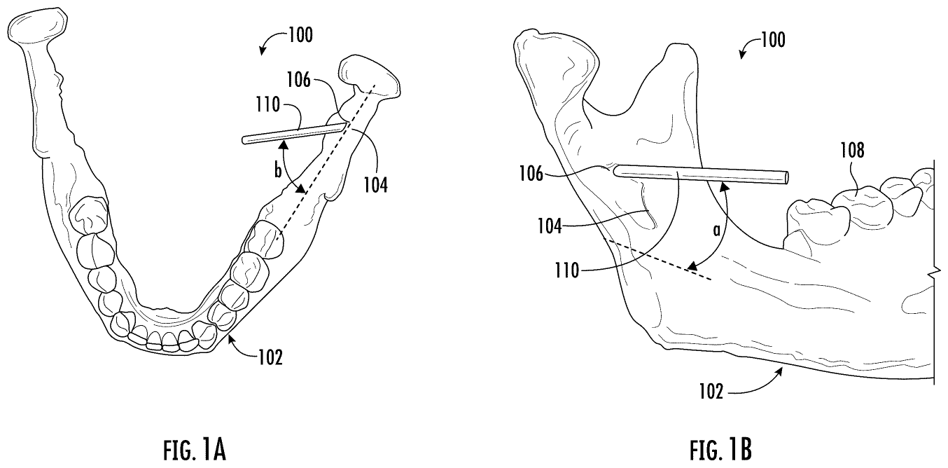

FIGS. 1A-1B illustrate a top plan view and a perspective view of a mandible represented by image data 100, in accordance with an implementation of the disclosure. Image data of a patient or prospective patient may be obtained through various methods to represent the anatomical structure of the patient in at least three dimensions within a computing environment. This rendering may be displayed on an end-point device.

A CT scanner may be implemented to obtain a first set of image data (i.e., CT scan data) by emitting X-rays circumferentially around the patient's head, collectively combining the images to provide a three-dimensional representation of the mandible and/or maxilla. The CT scan data may provide the bone structure. However, CT scans may not capture soft tissues and surface details with optimal precision. Accordingly, the present disclosure may also implement an optical scan, such as structured light or laser scanning, to obtain surface information as a second set of image data (i.e., optical scan data).

Additionally, or alternatively, Magnetic Resonance Imaging (“MRI”) techniques may be implemented to gather image data of a patient. MRI imaging devices may provide for the imaging of soft tissue of the patient (e.g., the oropharynx) and/or bone, and such image data received from an MRI imaging device may be used alone or in combination with CT scan(s) and/or optical scan(s). For example, in some implementations, image data from an MRI scan may be used in combination with image data from a CT scan. In other implementations, image data from a MRI scan may be used in combination with image data from an optical scan. In yet additional implementations, an MRI scan may be used alone, without the need to supplement that image data with image data from a CT scan, optical scan, or otherwise.

The first and second image data may be combined using computer software to obtain combined image data that represents not only the data collected by the optical scan data, but also from the CT scan data. By superimposing the optical scan data onto the CT scan data, a more comprehensive and accurate representation of the mandible and maxilla may be achieved. It shall be appreciated that in implementations where a single MRI, CT scan, optical scan, or otherwise is used to collect image data of both the soft tissue and bone, the first and second image data may already be part of the same dataset, and thus not need to be combined. Indeed, “combined image data” as used herein may refer to image data that contains image data of both soft tissue and bone, regardless of the source(s).

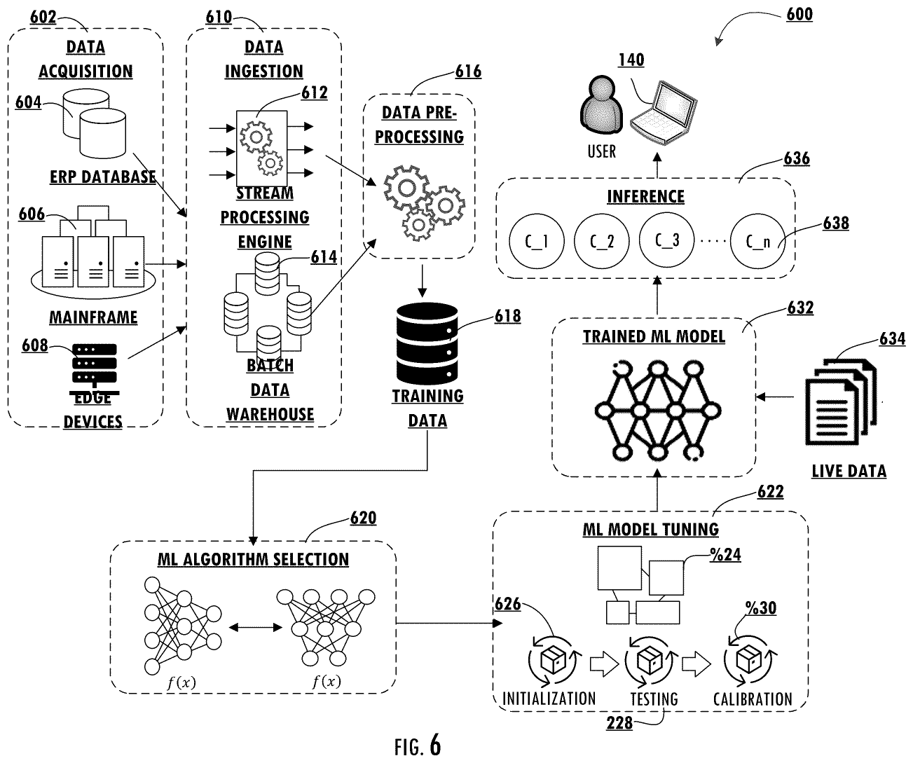

The combined image data 100 may be obtained by first identifying, either via the operator or a trained machine learning model 632 (as will be described in detail with respect to FIG. 6), at least one distinctive feature or anatomical point in both the first image data and the second image data that may be used as reference points. Then, translation, rotation, and/or scaling may be required to align the first image data with the second image data. The transformation of the image data is then applied to all the data available, which leads to combined image data 100. However, to fully integrate the first image data with the second image data, redundant data between the first and second image data may be removed from the combined image dataset.

Thus, an accurate representation of a patient's mandible via mandible data 102 and/or maxilla may be displayed on a computer interface, the combined image data 100 able to be manipulated for viewing and identifying various features of the patient's anatomy, including teeth data 108 and mandibular foramen data 104.

It shall be appreciated that the mandibular foramen is one structure in the mandible through which the mandibular nerve (i.e., the inferior alveolar nerve), a branch of the trigeminal nerve (cranial nerve V), passes. The mandibular nerve provides sensory innervation to the lower teeth, gums, and part of the tongue, as well as the skin of the lower face, and as such, the numbing of such areas may be required to comfortably perform various dental procedures. In the combined image data 100, the mandibular foramen is represented by the mandibular foramen data 104. For efficient and effective numbing of the lower teeth, gums, and part of the tongue, as well as the skin of the lower face, application of an anesthetic it optimally targeted for a target area 106 slightly above and proximate the entryway of the mandibular nerve into the mandibular foramen. As used herein with respect to the target area 106, “proximate” may refer to a boundary distance surrounding the centroid of the cross-section defining the entryway to the mandibular foramen. In some implementations, “proximate” the mandibular foramen may refer to between 0 and 2 mm from the centroid, between 0-3 mm from the centroid, 0-4 mm from the centroid, 0-5 mm from the centroid, 0-6 mm from the centroid, 0-7 mm from the centroid, 0-8 mm from the centroid, 0-9 mm from the centroid, 0-10 mm from the centroid, or 0-15 mm from the centroid.

A linear marker 110 may be introduced to the computer environment on the end-point device displaying the combined image data 100, such as to represent a straight needle that will ultimately be inserted into the patient and directed towards the target area. Accordingly, one end of the linear marker 110 may be positioned and fixed at the target area 106.

In some implementations, the length of the linear marker 110 may be predetermined based on a desired length of injection device (e.g., the length of a needle between the delivery tip and a larger portion of the needle and/or syringe). This length may vary, depending on the gauge of the needle used and the depth at which it needs to penetrate. However, it is contemplated that the present disclosure may accommodate needles of various lengths, including but not limited to lengths ranging from 6 mm to 40 mm, from 6 mm to 50 mm, from 20 mm to 50 mm, from 30 mm to 50 mm, from 40 mm to 50 mm, from 40 mm to 45 mm, or the like. Similarly, it is contemplated that the present disclosure may accommodate needles of various diameters/gauges, including but not limited to diameters/gauges ranging from 16 G-32 G. Accordingly, the diameter of the linear marker 110 may vary to represent the predetermined gauge of the needle.

Once a linear marker 110 of a predetermined length and diameter has been introduced to the computing environment on the end-point device, and the distal end (i.e., the fixed end) of the linear marker 110 has been applied to the target area 106, the free end (i.e., the proximate end) of the linear marker may be moved (e.g., rotated about the fixed end) in any number or directions to obtain a desired positioning.

For the delivery tip of a needle to reach the target area 106, the linear marker 110 generally extends from the target area 106 towards the midplane that bifurcates the right side of the mandible from the left side of the mandible. The elevation angle a must necessarily be sufficiently large such that a syringe (or other injection device coupled to the needle) will avoid interfering with the side of the mandible opposite that of the target area 106. In other words, if a needle is inserted to the target area 106 at a low elevation angle, the positioning of the syringe may be blocked by the teeth and/or jawbone of the patient on the side of the patient's jaw that is opposite the target area 106. Furthermore, it shall be appreciated that while the combined image data 100 provides for a detailed representation of a patient's mandible, soft tissues in the vicinity of a patient's mandible may not be present in the combined image data 100. Accordingly, soft tissues such as the buccal mucosa, oral commissure, tongue, oropharynx, uvula, and so forth may present obstacles to the proper positioning of a needle that are otherwise not shown in the computing environment. To avoid these soft tissues, the selection of angle b must be sufficiently large to allow access to the target area 106, while sufficiently small to prevent the interference with these soft tissues.

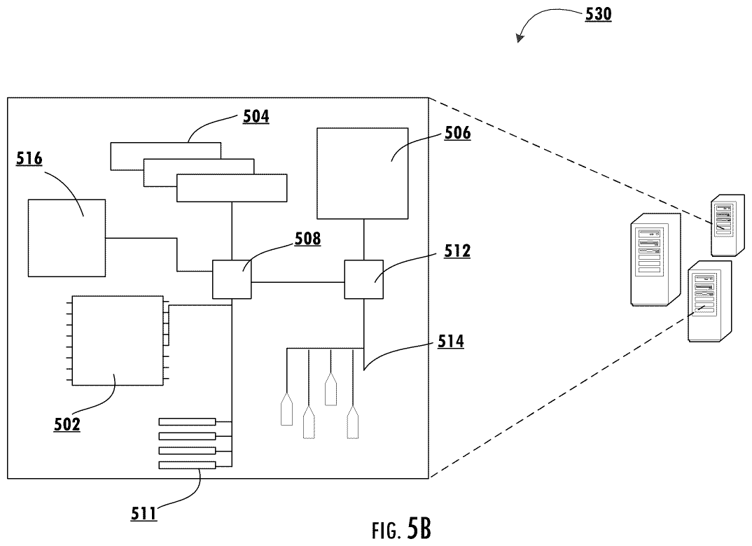

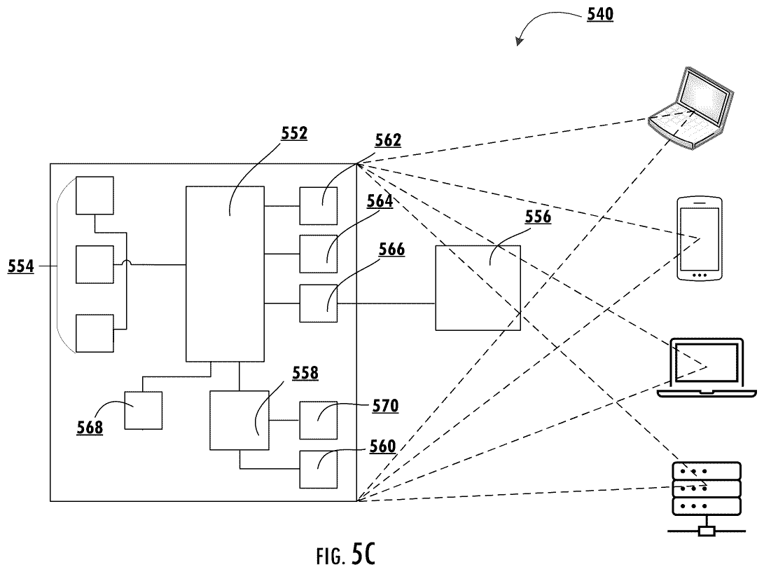

In some implementations, the determination of the orientation of the linear marker 110 described in the foregoing paragraphs is performed by an operator through an end-point device(s) 540 operatively coupled to system 530 (as will be described in detail with respect to FIG. 5), the operator having experience to determine the orientation most likely to be successful in reaching the target area 106 while also avoiding the soft tissue and other anatomical features.

In other implementations, determining the orientation of the linear marker 110 may include querying a trained machine learning model that has been trained using datasets including historical combined image data, historical anatomical landmark data (such as the positioning of the mandibular foramen or the mandibular condyle relative to the remainder of the mandible), and the corresponding orientation/positioning of the linear marker 110 identified to be a successful orientation/positioning in the dataset. In this way, the trained machine learning model 632 (as will be described in detail with respect to FIG. 6), having been trained on such training data, outputs the most likely successful orientation of the linear marker 110 to avoid the anatomical features.

Indeed, additionally, or alternatively, the machine learning model described in FIG. 6, may be implemented to make design selections based on labeled datasets of previous patient image data, including determination of linear marker orientation and position, fixed hub orientation, position, and geometrical features, aperture orientation, position, and geometrical features, needle support orientation, position, and geometrical features, and/or elongate member orientation, position, and geometrical features.

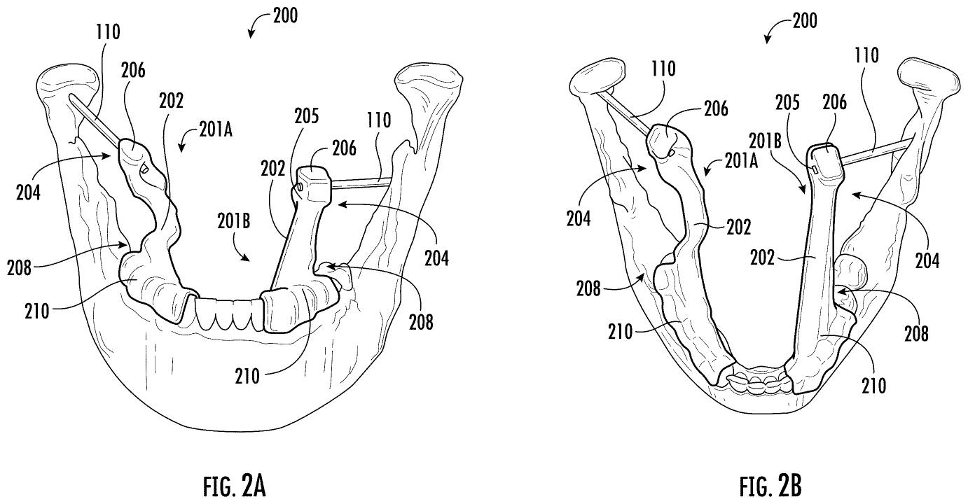

FIGS. 2A-2B illustrate a front view and a top plan view of a mandible represented by image data and a corresponding dental guide, in accordance with an implementation of the disclosure. It will be appreciated that FIGS. 2A and 2B each illustrate dental guides for two different injection targets. Specifically, the first dental guide rendering data 201A in FIGS. 2A and 2B has been formed to target the neck of the mandibular condyle. The second dental guide rendering data 201B in FIGS. 2A and 2B has been formed to target the mandibular foramen along the inner surface of the mandible.

Once the position and orientation of the linear marker 110 has been determined, dental guide rendering data may begin to be generated. Ultimately, this dental guide rendering data may be used to fabricate the detail guide through various methods of fabrication, as will be discussed in greater detail herein.

Although the following series of steps are described in a chronological order, it shall be appreciated that various other chronological orders of operation of these steps are also acceptable and result in dental guides of equal utility.

First, needle support rendering data may be generated to determine the size and location of the needle support 206 relative to the patient's mandible. The purpose of the needle support 206 is to allow for a needle to penetrate therethrough, and follow the linear path defined in the computing environment by the linear marker 110 such that the delivery tip of the needle reaches the target area 106. Moreover, the needle support 206 serves as a stopper to prevent the over-insertion of a needle into the patient, while also allowing the operator of the needle to continue insertion of the needle until the proximal end of the needle (generally having a threaded or luer lock style connector or the like) reaches the needle support 206 and is unable to proceed any further towards the target area 106.

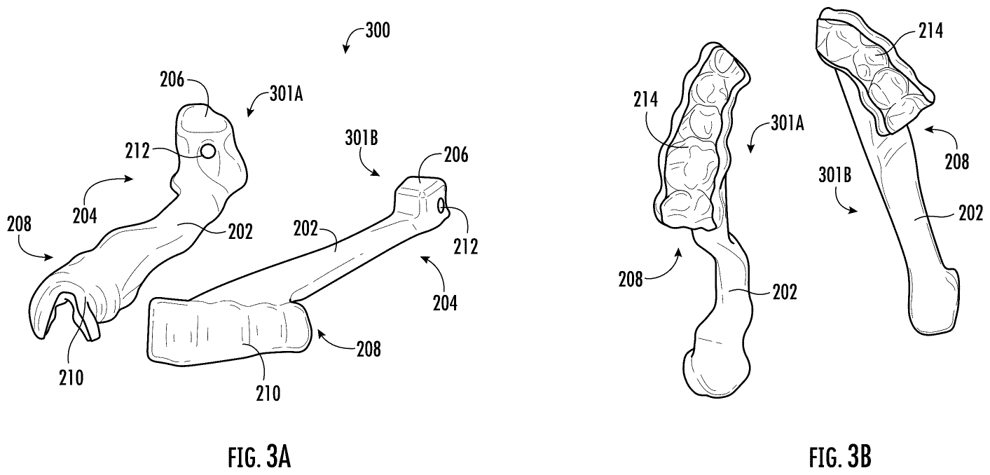

Turning now to FIGS. 3A-3B, which illustrate a perspective view and a bottom plan view of a pair of dental guides, in accordance with an implementation of the disclosure, the needle support 206 is illustrated having an aperture 212. This aperture is defined by a longitudinal axis substantially collinear to the longitudinal axis of the linear marker 110 within the computing environment. As such, the aperture may have a diameter equal to or greater than that of a needle passing therethrough. As will be described in greater detail herein, in some implementations a bushing or cylindrical insert may be provided in the dental guide to receive the needle with a level of precision and strength not otherwise found in typical apertures. Accordingly, the diameter of aperture 212 may be such as to accept said bushing or cylindrical insert.

Returning now to FIGS. 2A-2B, the needle support rendering data may similarly define an aperture 212 within the needle support rendering data. The needle support rendering data may also define a lingual surface 205. The lingual surface 205 is a surface proximate the portion of the aperture 212 closest to the tongue (i.e., the portion of the aperture that initially receives the needle). The lingual surface 205 is substantially co-planar to the free end of the linear marker 110 in the computing environment. In this way, a needle inserted into the aperture will “bottom out” against the lingual surface 205 when the delivery tip of the needle is proximate the target area.

Next, fixed hub rendering data may be generated that will ultimately correspond to the fixed hub 210 of the dental guide. While the needle support 206 may define a first end 204 of the dental guide, the fixed hub 210 may define a second end 208 of the dental guide, or vice versa. The fixed hub 210 provides a fixation point between the dental guide and the patient's mandible, thereby maintaining the free end of the dental guide (i.e., the needle support 206) in the desired location. To do so, the fixed hub 210 is attached to known anatomical features of the patient's mandible, generally at least one tooth.

Indeed, the combined image data 100 in the computing environment will depict at least one tooth of the mandible. The fixed hub rendering data is then generated by choosing the at least one tooth as a first surface, translating and/or scaling the first surface in a generally upward and outward manner, then connecting the first and second surfaces to form a solid body. Thus, the curved features and structure of the at least one tooth are transferred to the portion of the fixed hub 210 that will be attached to the at least one tooth during use of the dental guide. It shall be appreciated that in some implementations, the data in the combined image data 100 of one tooth may be used to generate the fixed hub rendering data. In other implementations, data in the combined image data 100 of two, three, four, five, or any number of teeth may be used to generate the fixed hub rendering data.

Turning now to FIG. 3B, the curved features, tooth contours, and structure of the at least one tooth are shown as contoured portion 214. In some implementations, the contoured portion 214 may comprise a material different from that of the remainder of the fixed hub 210. For example, the contoured portion 214 may be a softer rubber or pliable material that assists in the fixed hub 210 “sticking” to the at least on tooth. In other implementations, the contoured portion 214 may be the same material as the rest of the fixed hub 210.

Additionally, or alternatively, the fixed hub 210 (e.g., the contoured portion 214) may be affixed to the at least one tooth of the patient via use of a temporary adhesive, thereby allowing for the use of the dental guide without risk of movement of the dental guide or detachment of the dental guide from the at least one tooth. In doing so, the application of anesthetic may be more accurately positioned. Examples of temporary adhesive include, but are not limited to, zinc oxide-eugenol, glass ionomer, polycarboxylate, resin-based cements, or any other dental adhesive.

Returning back to FIGS. 2A-2B, elongate member rendering data may be generated for an elongate member extending between the needle support and the fixed hub (or, in the computing environment, the needle support rendering data and the fixed hub rendering data). The structure, dimensions, or features of the elongate member are generally not crucial to the functioning of the dental guide. However, the elongate member rendering data may be generated to avoid anatomical features of the patient, or any oral devices worn by the patient. For example, the elongate member rendering data in some implementations may connect the needle support to the fixed hub via a linear path. However, in some implementations, a curvilinear path may be ideal to avoid the tongue, protruding teeth, and so forth. Additionally, or alternatively, some dental guides implementations (for example, those that target the neck of the mandibular condyle) may include extending the elongate member data medially from the fixed hub data away from the lingual surface of the teeth (i.e., curved inwards towards the tongue). Additionally, or alternatively, some implementations may include extending the elongate member data laterally from the fixed hub data, or other portions of the elongate member data, towards the buccal surface of the teeth (i.e., curved outwards away from the tongue).

While FIGS. 2A-2B illustrate the generation of a pair of dental guides via first dental guide rendering data 201A and second dental guide rendering data 201B, it shall be appreciated that the foregoing steps may be performed on one portion of a patient's mandible (e.g., the left side or right side) to obtain a single dental guide, or both portions of a patient's mandible simultaneously or in series (e.g., both the left side and the right side) to obtain the pair of dental guides. In implementations where the pair of dental guides is to be generated, the system 530 may take into consideration the structure of the dental guide rendering data on the opposite side to that which is being generated in order to position and orient the linear marker 110 to avoid interference with the dental guide on the opposite side during use.

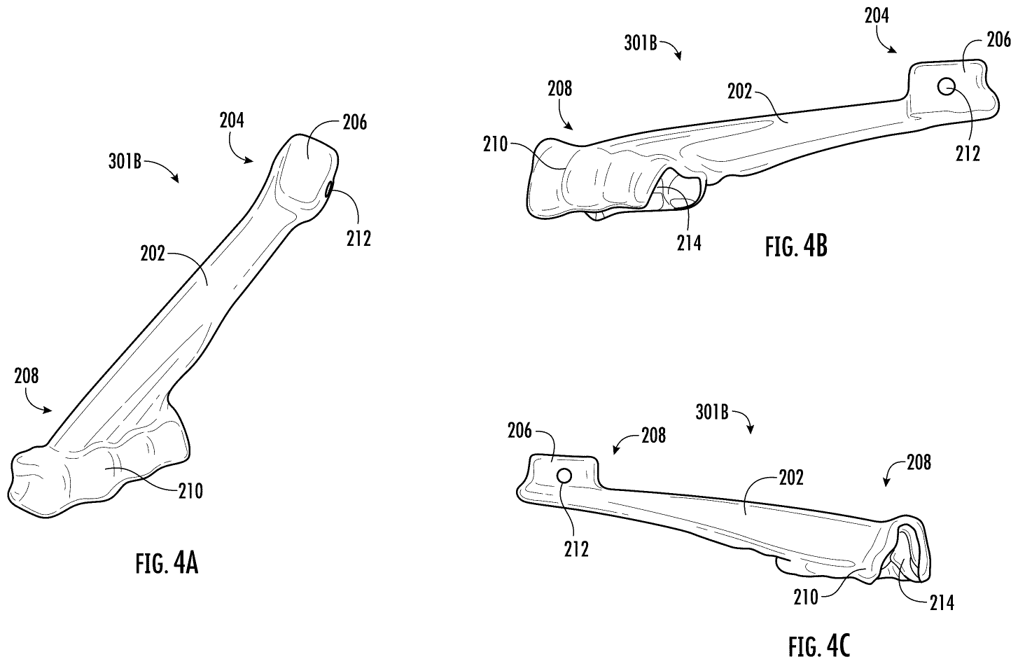

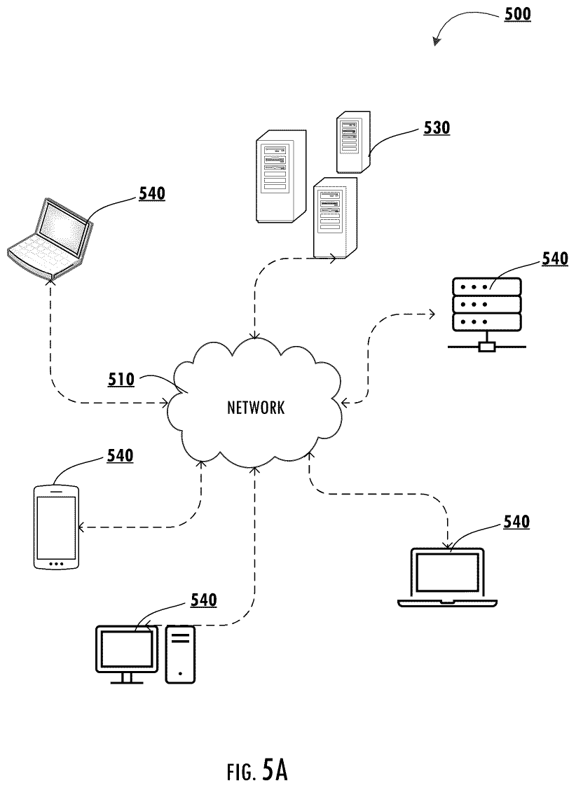

FIGS. 4A-4C illustrate a perspective view, a left side view, and a right side view of a dental guide, in accordance with an implementation of the disclosure. Once the dental guide rendering data has been generated and combined (i.e., the combined rendering data between the fixed hub rendering data, the needle support rendering data, and the elongate member rendering data), the dental guide rendering data may be transformed into additive-manufacturing ready data by converting the rendering data to STL file(s), STEP file(s), or the like. This data may then be used in additive manufacturing processes to fabricate the dental guide 301B from the dental guide rendering data. Examples of additive manufacturing processes include, but are not limited to, stereolithography (“SLA”), selective laser sintering (“SLS”), fused deposition modeling (“FDM”), binder jetting, electron beam melting, Digital Light Processing (“DLP”), material jetting, or the like. Additionally, or alternatively, the rendering data may be used for reductive (i.e., subtractive) manufacturing of the dental guide 301B from one or more base materials, which may include CNC machining, milling, drilling, turning, grinding, electrical discharge machining (“EDM”), waterjet cutting, plasma cutting, sawing, broaching, and reaming or the like, or various combinations thereof. Alternatively, this data may be used for other manufacturing methods like CNC machining a block of material for injection molding of the dental guide(s). Additionally, or alternatively, the dental guide(s) using the aforementioned processes may be used to create molds (such as silicone molds) for lower-cost repeatable molding using various resins for materials otherwise not available for additive manufacturing.

In any case, materials for the dental guide(s) may include, but are not limited to, thermoplastic polymers such as acrylonitrile butadiene styrene (ABS), polylactic acid (PLA), polyamide (nylon), polyethylene terephthalate (PET), and polyether ether ketone (PEEK), ethylene vinyl acetate (EVA), polyethylene, polypropylene, polyurethane, silicone, latex, rubber, vinyl, acrylic, and combinations thereof, thermoplastic elastomers (TPE), polyvinyl acetate (PVA), or nanocomposite materials, metals such as aluminum, titanium, stainless steel, inconel, cobalt-chromium, and copper alloys, ceramics, including zirconia and alumina, and/or composite materials such as carbon fibers, glass fibers, or Kevlar with polymers. Additionally, or alternatively, polymers may include resins suitable for light-curing during additive manufacturing processes. Any of the aforementioned materials may be implemented with specialized formulations intended for use with the human body, including dental-specific, surgical-specific, bio-compatible, and/or food-grade resins.

As previously described, in some implementations the contoured portion 214 may comprise a material different than that of the fixed hub. Accordingly, in some implementations, the fabrication of the dental guide 301B may require multiple processing steps, such as one fabrication step to fabricate the entirety of the dental guide 301B other than the contoured portion 214, then a secondary fabrication step to fabricate and attach the contoured portion 214.

Furthermore, the aperture 212 of the dental guide 310B may include a cylindrical insert (e.g., a bushing) with an internal diameter for receiving a needle. The cylindrical insert may be formed of any number of materials, including metals such as aluminum, titanium, stainless steel, inconel, cobalt-chromium, and copper alloys, plastics, composites, or the like. The cylindrical insert may be inserted into the aperture 212 after fabrication thereof, and subsequently secured by gluing, heat-pressing, melting, ultrasonic welding, and so forth. However, in some implementations, the cylindrical insert may be provided to the aperture before or during the fabrication, such as to capture the cylindrical insert without requiring additional steps to secure the cylindrical insert.

The inner diameter of the of the cylindrical insert may be equal to or greater than the needle of which it is predetermined to receive. It shall be appreciated that precision fitting between the cylindrical insert and the needle leads to a more repeatable and precise alignment of the needle with the target area 106. Accordingly, tolerances between the inner diameter of the cylindrical insert and the outer diameter of the needle may be +/−0.0001 in., between +/−0.0001 in. and +/−0.0005 in., between +/−0.0005 in. and +/−0.001 in., between +/−0.001 in. and +/−0.005 in., between +/−0.005 in. and +/−0.01 in, between +/−0.01 in. and +/−0.05 in, or between +/−0.05 in. and +/−0.1 in.

While the aforementioned generation and fabrication processes are directed to dental guide(s) specific to an individual patient, based on a unique mandible and location of the anatomical landmark(s), the foregoing processes may be used to generate a set of dental guides that are non-specific to any individual patient. After the foregoing processes have been performed numerous times over a variety of patients, historical needle support rendering data may be available sufficient to categorize patients into a specific group of a predetermined number of groups based on features of the mandible such as mandible dimensions, teeth location, or the like. Once these features have been determined, often without the need for CT image data, optical scan image data, or other types of image data gathering techniques, a trained operator will be able to select one of several pre-fabricated dental guides. The fixed hub and corresponding contoured portion 214 may be generally curved in shape with a trough for receiving at least one tooth of the patient. While the use of such dental guides may lead to a delivery tip slightly off-target from the target area, the delivery tip location is still generally closer to the target area than when using traditional anesthesia methods without a dental guide, leading to increased patient comfort.

FIGS. 5A-5C illustrate technical components of an exemplary distributed computing environment 500 for generating a dental guide, in accordance with an implementation of the disclosure. As shown in FIG. 5A, the distributed computing environment 500 contemplated herein may include a system 530, an end-point device(s) 540, and a network 510 over which the system 530 and end-point device(s) 540 communicate therebetween. FIG. 5A illustrates only one example of an implementation of the distributed computing environment 500, and it will be appreciated that in other implementations one or more of the systems, devices, and/or servers may be combined into a single system, device, or server, or be made up of multiple systems, devices, or servers. Also, the distributed computing environment 500 may include multiple systems, same or similar to system 530, with each system providing portions of the necessary operations (e.g., as a server bank, a group of blade servers, or a multi-processor system).

In some implementations, the system 530 and the end-point device(s) 540 may have a client-server relationship in which the end-point device(s) 540 are remote devices that request and receive service from a centralized server, i.e., the system 530. In some other implementations, the system 530 and the end-point device(s) 540 may have a peer-to-peer relationship in which the system 530 and the end-point device(s) 540 are considered equal and all have the same abilities to use the resources available on the network 510. Instead of having a central server (e.g., system 530) which would act as the shared drive, each device that is connect to the network 510 would act as the server for the files stored on it.

The system 530 may represent various forms of servers, such as web servers, database servers, file server, or the like, various forms of digital computing devices, such as laptops, desktops, video recorders, audio/video players, radios, workstations, or the like, or any other auxiliary network devices, such as wearable devices, Internet-of-things devices, electronic kiosk devices, entertainment consoles, mainframes, or the like, or any combination of the aforementioned.

The end-point device(s) 540 may represent various forms of electronic devices, including user input devices such as personal digital assistants, cellular telephones, smartphones, laptops, desktops, and/or the like, merchant input devices such as point-of-sale (POS) devices, electronic payment kiosks, and/or the like, electronic telecommunications device (e.g., automated teller machine (ATM)), and/or edge devices such as routers, routing switches, integrated access devices (IAD), and/or the like.

The network 510 may be a distributed network that is spread over different networks. This provides a single data communication network, which can be managed jointly or separately by each network. Besides shared communication within the network, the distributed network often also supports distributed processing. The network 510 may be a form of digital communication network such as a telecommunication network, a local area network (“LAN”), a wide area network (“WAN”), a global area network (“GAN”), the Internet, or any combination of the foregoing. The network 510 may be secure and/or unsecure and may also include wireless and/or wired and/or optical interconnection technology.

It is to be understood that the structure of the distributed computing environment and its components, connections and relationships, and their functions, are meant to be exemplary only, and are not meant to limit implementations of the disclosures described and/or claimed in this document. In one example, the distributed computing environment 500 may include more, fewer, or different components. In another example, some or all of the portions of the distributed computing environment 500 may be combined into a single portion or all of the portions of the system 530 may be separated into two or more distinct portions.

FIG. 5B illustrates an exemplary component-level structure of the system 530, in accordance with an implementation of the disclosure. As shown in FIG. 5B, the system 530 May include a processor 502, memory 504, input/output (I/O) device 516, and a storage device 506. The system 530 may also include a high-speed interface 508 connecting to the memory 504, and a low-speed interface 512 connecting to low-speed bus 514 and storage device 506. Each of the components 502, 504, 508, 510, and 512 may be operatively coupled to one another using various buses and may be mounted on a common motherboard or in other manners as appropriate. As described herein, the processor 502 may include a number of subsystems to execute the portions of processes described herein. Each subsystem may be a self-contained component of a larger system (e.g., system 530) and capable of being configured to execute specialized processes as part of the larger system.

The processor 502 can process instructions, such as instructions of an application that may perform the functions disclosed herein. These instructions may be stored in the memory 504 (e.g., non-transitory storage device) or on the storage device 506, for execution within the system 530 using any subsystems described herein. It is to be understood that the system 530 may use, as appropriate, multiple processors, along with multiple memories, and/or I/O devices, to execute the processes described herein.

The memory 504 stores information within the system 530. In one implementation, the memory 504 is a volatile memory unit or units, such as volatile random access memory (RAM) having a cache area for the temporary storage of information, such as a command, a current operating state of the distributed computing environment 500, an intended operating state of the distributed computing environment 500, instructions related to various methods and/or functionalities described herein, and/or the like. In another implementation, the memory 504 is a non-volatile memory unit or units. The memory 504 may also be another form of computer-readable medium, such as a magnetic or optical disk, which may be embedded and/or may be removable. The non-volatile memory may additionally or alternatively include an EEPROM, flash memory, and/or the like for storage of information such as instructions and/or data that may be read during execution of computer instructions. The memory 504 may store, recall, receive, transmit, and/or access various files and/or information used by the system 530 during operation.

The storage device 506 is capable of providing mass storage for the system 530. In one aspect, the storage device 506 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier may be a non-transitory computer- or machine-readable storage medium, such as the memory 504, the storage device 506, or memory on processor 502.

The high-speed interface 508 manages bandwidth-intensive operations for the system 530, while the low-speed controller 512 manages lower bandwidth-intensive operations. Such allocation of functions is exemplary only. In some implementations, the high-speed interface 508 is coupled to memory 504, input/output (I/O) device 516 (e.g., through a graphics processor or accelerator), and to high-speed expansion ports 511, which may accept various expansion cards (not shown). In such an implementation, low-speed controller 512 is coupled to storage device 506 and low-speed expansion port 514. The low-speed expansion port 514, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The system 530 may be implemented in a number of different forms. For example, the system 530 may be implemented as a standard server, or multiple times in a group of such servers. Additionally, the system 530 may also be implemented as part of a rack server system or a personal computer such as a laptop computer. Alternatively, components from system 530 may be combined with one or more other same or similar systems and an entire system 530 may be made up of multiple computing devices communicating with each other.

FIG. 5C illustrates an exemplary component-level structure of the end-point device(s) 540, in accordance with an implementation of the disclosure. As shown in FIG. 5C, the end-point device(s) 540 includes a processor 552, memory 554, an input/output device such as a display 556, a communication interface 558, and a transceiver 560, among other components. The end-point device(s) 540 may also be provided with a storage device, such as a microdrive or other device, to provide additional storage. Each of the components 552, 554, 558, and 560, are interconnected using various buses, and several of the components may be mounted on a common motherboard or in other manners as appropriate.

The processor 552 is configured to execute instructions within the end-point device(s) 540, including instructions stored in the memory 554, which in one implementation includes the instructions of an application that may perform the functions disclosed herein, including certain logic, data processing, and data storing functions. The processor may be implemented as a chipset of chips that include separate and multiple analog and digital processors. The processor may be configured to provide, for example, for coordination of the other components of the end-point device(s) 540, such as control of user interfaces, applications run by end-point device(s) 540, and wireless communication by end-point device(s) 540.

The processor 552 may be configured to communicate with the user through control interface 564 and display interface 566 coupled to a display 556. The display 556 may be, for example, a TFT LCD (Thin-Film-Transistor Liquid Crystal Display) or an OLED (Organic Light Emitting Diode) display, or other appropriate display technology. The display 556 may comprise appropriate circuitry and configured for driving the display 556 to present graphical and other information to a user. The control interface 564 may receive commands from a user and convert them for submission to the processor 552. In addition, an external interface 568 may be provided in communication with processor 552, so as to enable near area communication of end-point device(s) 540 with other devices. External interface 568 may provide, for example, for wired communication in some implementations, or for wireless communication in other implementations, and multiple interfaces may also be used.

The memory 554 stores information within the end-point device(s) 540. The memory 554 can be implemented as one or more of a computer-readable medium or media, a volatile memory unit or units, or a non-volatile memory unit or units. Expansion memory may also be provided and connected to end-point device(s) 540 through an expansion interface (not shown), which may include, for example, a SIMM (Single In Line Memory Module) card interface. Such expansion memory may provide extra storage space for end-point device(s) 540 or may also store applications or other information therein. In some implementations, expansion memory may include instructions to carry out or supplement the processes described above and may include secure information also. For example, expansion memory may be provided as a security module for end-point device(s) 540 and may be programmed with instructions that permit secure use of end-point device(s) 540. In addition, secure applications may be provided via the SIMM cards, along with additional information, such as placing identifying information on the SIMM card in a non-hackable manner.

The memory 554 may include, for example, flash memory and/or NVRAM memory. In one aspect, a computer program product is tangibly embodied in an information carrier. The computer program product contains instructions that, when executed, perform one or more methods, such as those described herein. The information carrier is a computer- or machine-readable medium, such as the memory 554, expansion memory, memory on processor 552, or a propagated signal that may be received, for example, over transceiver 560 or external interface 568.

In some implementations, the user may use the end-point device(s) 540 to transmit and/or receive information or commands to and from the system 530 via the network 510. Any communication between the system 530 and the end-point device(s) 540 may be subject to an authentication protocol allowing the system 530 to maintain security by permitting only authenticated users (or processes) to access the protected resources of the system 530, which may include servers, databases, applications, and/or any of the components described herein. To this end, the system 530 may trigger an authentication subsystem that may require the user (or process) to provide authentication credentials to determine whether the user (or process) is eligible to access the protected resources. Once the authentication credentials are validated and the user (or process) is authenticated, the authentication subsystem may provide the user (or process) with permissioned access to the protected resources. Similarly, the end-point device(s) 540 may provide the system 530 (or other client devices) permissioned access to the protected resources of the end-point device(s) 540, which may include a GPS device, an image capturing component (e.g., camera), a microphone, and/or a speaker.

The end-point device(s) 540 may communicate with the system 530 through communication interface 558, which may include digital signal processing circuitry where necessary. Communication interface 558 may provide for communications under various modes or protocols, such as the Internet Protocol (IP) suite (commonly known as TCP/IP). Protocols in the IP suite define end-to-end data handling methods for everything from packetizing, addressing and routing, to receiving. Broken down into layers, the IP suite includes the link layer, containing communication methods for data that remains within a single network segment (link); the Internet layer, providing internetworking between independent networks; the transport layer, handling host-to-host communication; and the application layer, providing process-to-process data exchange for applications. Each layer contains a stack of protocols used for communications. In addition, the communication interface 558 may provide for communications under various telecommunications standards (2G, 3G, 4G, 5G, and/or the like) using their respective layered protocol stacks. These communications may occur through a transceiver 560, such as radio-frequency transceiver. In addition, short-range communication may occur, such as using a Bluetooth, Wi-Fi, or other such transceiver (not shown). In addition, GPS (Global Positioning System) receiver module 570 may provide additional navigation- and location-related wireless data to end-point device(s) 540, which may be used as appropriate by applications running thereon, and in some implementations, one or more applications operating on the system 530.

The end-point device(s) 540 may also communicate audibly using audio codec 562, which may receive spoken information from a user and convert the spoken information to usable digital information. Audio codec 562 may likewise generate audible sound for a user, such as through a speaker, e.g., in a handset of end-point device(s) 540. Such sound may include sound from voice telephone calls, may include recorded sound (e.g., voice messages, music files, etc.) and may also include sound generated by one or more applications operating on the end-point device(s) 540, and in some implementations, one or more applications operating on the system 530.

Various implementations of the distributed computing environment 500, including the system 530 and end-point device(s) 540, and techniques described here can be realized in digital electronic circuitry, integrated circuitry, specially designed ASICs (application specific integrated circuits), computer hardware, firmware, software, and/or combinations.

FIG. 6 illustrates an exemplary machine learning (ML) subsystem architecture 600, in accordance with an implementation of the disclosure. The machine learning subsystem 600 may include a data acquisition engine 602, data ingestion engine 610, data pre-processing engine 616, ML model tuning engine 622, and inference engine 636.

The data acquisition engine 602 may identify various internal and/or external data sources to generate, test, and/or integrate new features for training the machine learning model 624. These internal and/or external data sources 604, 606, and 608 may be initial locations where the data originates or where physical information is first digitized. The data acquisition engine 602 may identify the location of the data and describe connection characteristics for access and retrieval of data. In some implementations, data is transported from each data source 604, 606, or 608 using any applicable network protocols, such as the File Transfer Protocol (FTP), Hyper-Text Transfer Protocol (HTTP), or any of the myriad Application Programming Interfaces (APIs) provided by websites, networked applications, and other services. In some implementations, the these data sources 604, 606, and 608 may include Enterprise Resource Planning (ERP) databases that host data related to day-to-day business activities such as accounting, procurement, project management, exposure management, supply chain operations, and/or the like, mainframe that is often the entity's central data processing center, edge devices that may be any piece of hardware, such as sensors, actuators, gadgets, appliances, or machines, that are programmed for certain applications and can transmit data over the internet or other networks, and/or the like. The data acquired by the data acquisition engine 602 from these data sources 604, 606, and 608 may then be transported to the data ingestion engine 610 for further processing.

Depending on the nature of the data imported from the data acquisition engine 602, the data ingestion engine 610 may move the data to a destination for storage or further analysis. Typically, the data imported from the data acquisition engine 602 may be in varying formats as they come from different sources, including RDBMS, other types of databases, S3 buckets, CSVs, or from streams. Since the data comes from different places, it needs to be cleansed and transformed so that it can be analyzed together with data from other sources. At the data acquisition engine 602, the data may be ingested in real-time, using the stream processing engine 612, in batches using the batch data warehouse 614, or a combination of both. The stream processing engine 612 may be used to process continuous data stream (e.g., data from edge devices), i.e., computing on data directly as it is received, and filter the incoming data to retain specific portions that are deemed useful by aggregating, analyzing, transforming, and ingesting the data. On the other hand, the batch data warehouse 614 collects and transfers data in batches according to scheduled intervals, trigger events, or any other logical ordering.

In machine learning, the quality of data and the useful information that can be derived therefrom directly affects the ability of the machine learning model 624 to learn. The data pre-processing engine 616 may implement advanced integration and processing steps needed to prepare the data for machine learning execution. This may include modules to perform any upfront, data transformation to consolidate the data into alternate forms by changing the value, structure, or format of the data using generalization, normalization, attribute selection, and aggregation, data cleaning by filling missing values, smoothing the noisy data, resolving the inconsistency, and removing outliers, and/or any other encoding steps as needed.

In addition to improving the quality of the data, the data pre-processing engine 616 may implement feature extraction and/or selection techniques to generate training data 618. Feature extraction and/or selection is a process of dimensionality reduction by which an initial set of data is reduced to more manageable groups for processing. A characteristic of these large data sets is a large number of variables that require a lot of computing resources to process. Feature extraction and/or selection may be used to select and/or combine variables into features, effectively reducing the amount of data that must be processed, while still accurately and completely describing the original data set. Depending on the type of machine learning algorithm being used, this training data 618 may require further enrichment. For example, in supervised learning, the training data is enriched using one or more meaningful and informative labels to provide context so a machine learning model can learn from it. For example, image data of previous patients may be labeled with soft tissue location and/or parameters such as curvature of the jawbone, length and width of jawbone, height and depth of dental arches, arch form curvature, alveolar ridge height, mandibular angle, angle of tooth incline, and distances between anatomical landmarks such as mandibular condyles. Additionally, or alternatively, for at least a portion of the previous patient image data to be used as training data, the orientation of a linear marker, the size and orientation of needle support (with or without an aperture), fixed hub size and orientation, and/or curvature or other geometric features of the elongate member may accompany the label(s) and be used to infer the geometric features and positioning of the linear marker, needle support, fixed hub, and/or elongate member. Data labeling is required for a variety of use cases including computer vision, natural language processing, and speech recognition. In contrast, unsupervised learning uses unlabeled data to find patterns in the data, such as inferences or clustering of data points. In some implementations, the foregoing previous patient image data for machine learning model training may be vectorized in two-dimensional or three-dimensional space to improve the accuracy of pattern recognition and feature detection within the model.

The ML model tuning engine 622 may be used to train a machine learning model 624 using the training data 618 to make predictions or decisions without explicitly being programmed to do so. The machine learning model 624 represents what was learned by the selected machine learning algorithm 620 and represents the rules, numbers, and any other algorithm-specific data structures required for classification. Selecting the right machine learning algorithm may depend on a number of different factors, such as the problem statement and the kind of output needed, type and size of the data, the available computational time, number of features and observations in the data, and/or the like. Machine learning algorithms may refer to programs (math and logic) that are configured to self-adjust and perform better as they are exposed to more data. To this extent, machine learning algorithms are capable of adjusting their own parameters, given feedback on previous performance in making prediction about a dataset.

The machine learning algorithms contemplated, described, and/or used herein include supervised learning (e.g., using logistic regression, using back propagation neural networks, using random forests, decision trees, etc.), unsupervised learning (e.g., using an Apriori algorithm, using K-means clustering), semi-supervised learning, reinforcement learning (e.g., using a Q-learning algorithm, using temporal difference learning), and/or any other suitable machine learning model type. Each of these types of machine learning algorithms can implement any of one or more of a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolution network method, a stacked auto-encoder method, etc.), a dimensionality reduction method (e.g., principal component analysis, partial least squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and/or the like.

To tune the machine learning model, the ML model tuning engine 622 may repeatedly execute cycles of experimentation 626, testing 628, and tuning 630 to optimize the performance of the machine learning algorithm 620 and refine the results in preparation for deployment of those results for consumption or decision making. To this end, the ML model tuning engine 622 may dynamically vary hyperparameters each iteration (e.g., number of trees in a tree-based algorithm or the value of alpha in a linear algorithm), run the algorithm on the data again, then compare its performance on a validation set to determine which set of hyperparameters results in the most accurate model. The accuracy of the model is the measurement used to determine which set of hyperparameters is best at identifying relationships and patterns between variables in a dataset based on the input, or training data 618. A fully trained machine learning model 632 is one whose hyperparameters are tuned and model accuracy maximized.

The trained machine learning model 632, similar to any other software application output, can be persisted to storage, file, memory, or application, or looped back into the processing component to be reprocessed. More often, the trained machine learning model 632 is deployed into an existing production environment to make practical business decisions based on live data 634. To this end, the machine learning subsystem 600 uses the inference engine 636 to make such decisions. The type of decision-making may depend upon the type of machine learning algorithm used. For example, machine learning models trained using supervised learning algorithms may be used to structure computations in terms of categorized outputs (e.g., C_1, C_2 . . . . C_n 638) or observations based on defined classifications, represent possible solutions to a decision based on certain conditions, model complex relationships between inputs and outputs to find patterns in data or capture a statistical structure among variables with unknown relationships, and/or the like. On the other hand, machine learning models trained using unsupervised learning algorithms may be used to group (e.g., C_1, C_2 . . . . C_n 638) live data 634 based on how similar they are to one another to solve exploratory challenges where little is known about the data, provide a description or label (e.g., C_1, C_2 . . . . C_n 638) to live data 634, such as in classification, and/or the like. These categorized outputs, groups (clusters), or labels are then presented to the user input system 130. In still other cases, machine learning models that perform regression techniques may use live data 634 to predict or forecast continuous outcomes.

It will be understood that the implementation of the machine learning subsystem 600 illustrated in FIG. 6 is exemplary and that other implementations may vary. As another example, in some implementations, the machine learning subsystem 600 may include more, fewer, or different components.

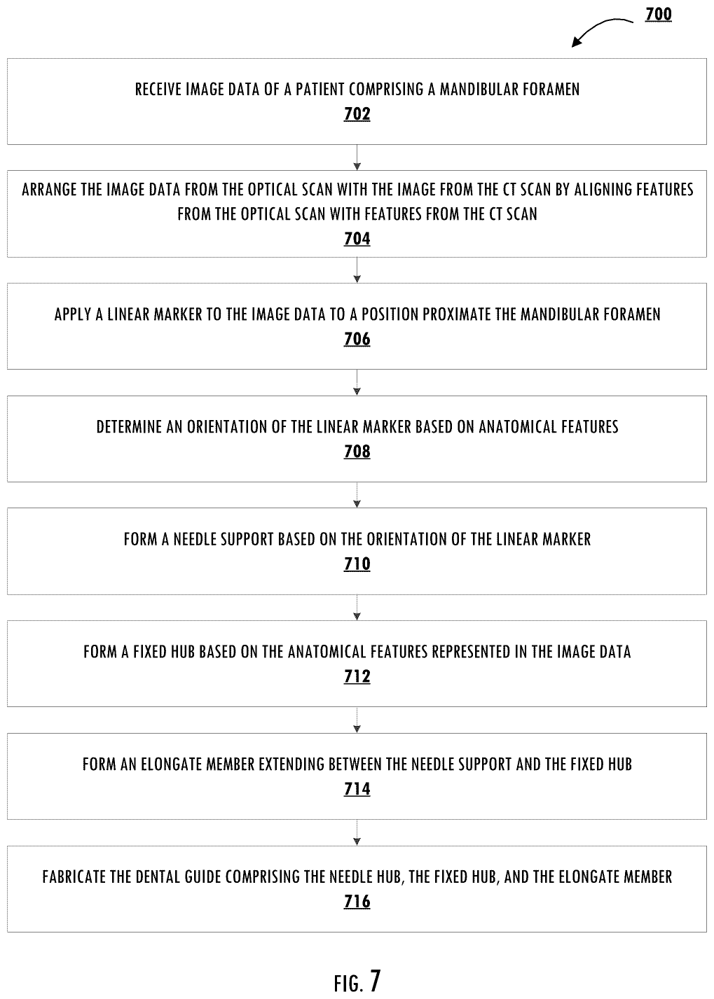

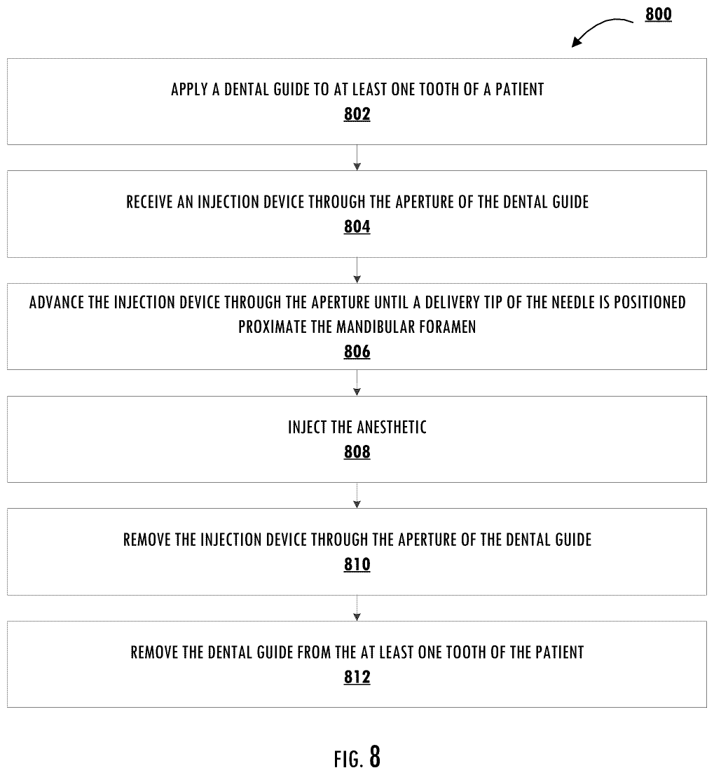

FIG. 7 illustrates a process flow 700 of the generation of a dental guide, in accordance with an implementation of the disclosure. The process may begin at block 702, where the system 530 receives image data. The image data comprises a representation of the mandibular foramen of the patient. In some implementations, the image data may be from an optical scan, a CT scan, and/or an MRI image. As previously described with respect to FIGS. 1A-4C, the image data of the CT scan may be provided by a CT scanner that emits X-rays circumferentially around the patient's head, and collectively combines these images to provide a three-dimensional representation of the mandible and/or maxilla. The optical scan data may be obtained through structured light or laser scanning to obtain surface information. In some implementations, the system may only receive image data from an optical scan, a CT scan, or an MRI image. In such implementations, the process of block 704 may not be necessary.

At block 704, the image data from the optical scan, CT scan, and/or MRI image (i.e., any sources of the first and second image data that, when alone or combined, contain imaging data of soft tissue and bone) may be arranged on the system 530 to form image data that is combined, this image data being displayed on the end-point device 540. As previously described with respect to FIGS. 1A-4C, the image data may be obtained by first identifying (either via the operator at the end-point device 540 or a machine learning model 632 of the system 530) at least one distinctive feature or anatomical point in both the first image data and the second image data that may be used as reference points. Then, translation, rotation, and/or scaling may be required to align the first image data with the second image data. The transformation of the image data at the reference points is then applied to all the image data available. However, to fully integrate the first image data with the second image data, redundant data between the first and second image data may be removed from the image data.

Next, at block 706, a linear marker is applied to a target area in the image data. As previously described with respect to FIGS. 1A-4C, the size and length of the linear marker is predetermined based on the characteristics of a needle to be used. The target area and/or anatomical landmark is located. In some implementations, the machine learning model 632 is trained using training data to determine the location of the anatomical landmark and/or target area. For example, in supervised machine learning implementations, one or more image data of previous patients may be labeled or annotated with the presence of the anatomical landmark/target area, which may then be provided to the machine learning model 632. One end of the linear marker as displayed on the end-point device 540 is affixed to the target area. In some implementations, the machine learning model 632 is trained using training data to affix the linear marker to the target area.

At block 708, the orientation of the linear marker is determined based on the anatomical features of the patient. As previously described with respect to FIGS. 1A-4C, the orientation and position of the linear marker may be determined based on the location of soft tissue (generally not captured in the image data), jawbone location, and so forth. In some implementations, the machine learning model is trained using training data to provide a trained machine learning model 632 to determine the optimal position of the linear marker to avoid soft tissue of any patient with a high probability.

For example, the image data of previous patients may be labeled or otherwise annotated with soft tissue location (for that which is able to be captured in the image data), and/or parameters of jawbone and/or mandible features (e.g., curvature of the jawbone, length and width of jawbone, height and depth of dental arches, arch form curvature, alveolar ridge height, mandibular angle, angle of tooth incline, and distances between anatomical landmarks such as mandibular condyles). In such implementations, the orientation of the linear marker relative the previous patient image data may be used to infer the predicted linear marker orientation and/or position. For example, the X, Y, and Z coordinates may have been determined in the previous patient image data for each of the two ends of the linear marker, that, when connected with a line, form the linear marker. Additionally, or alternatively, the X, Y, and Z coordinates of one end of the linear marker along with the elevation angle a and angle b may have been determined in the previous patient image data. Other methods may also be used to determine relevant coordinates and angles to quantify the placement and/or alignment the linear marker, any of which may be provided to the machine learning model as training data.

At block 710, the needle support is formed (e.g., rendering data is generated) for the needle support based on the orientation and positioning of the linear marker. The position of the proximal end of the linear marker (e.g., the free end) is determined by the length of the needle to be received in the needle support. The needle support rendering data may be formed by first providing a lingual surface at the proximal end of the linear marker, then expanding the needle support rendering data in the upwards and downwards directions at predetermined distances, and in the direction parallel to the longitudinal axis of the linear marker for a predetermined distance. As previously described, in implementations where machine learning is implemented, the predetermined distances, including geometries of the surfaces of the needle support, corresponding thicknesses, or the like, may be inferred based on training data provided to the machine learning model.

The aperture may also be formed in the needle support rendering data, the aperture being a predetermined diameter for receiving a needle and/or a cylindrical insert and extending through the entirety of the needle support rendering data in the direction parallel to the linear marker. As previously described, in implementations where machine learning is implemented, the aperture location, diameter, and/or orientation may be inferred based on training data provided to the machine learning model.

Next, at block 712, the fixed hub is formed (e.g., rendering data for a fixed hub is generated) based on anatomical features such as at least one tooth. As previously described with respect to FIGS. 1A-4C, one or more surfaces of the at least one tooth or other anatomical features may be used as a basis for the fixed hub rendering data. In some implementations, the fixed hub rendering data is formed by translating and/or scaling these one or more surfaces in a generally upward and outward manner to create the surface of the contoured portion 214. Then, the contoured portion may be translated and/or scaled in a generally upward and outward manner to form the surface of the fixed hub opposite the contoured portion. The space between the contoured portion and the opposite surface is filled in as a solid body. In some implementations, the surface opposite the contoured portion is substantially smooth with at least one flat surface. Additionally, or alternatively, the contoured portion 214 may be substantially smooth with at least one flat surface. As previously described, in implementations where machine learning is implemented, the space between the contoured portion and the opposite surface, translation or scaling amount, and other geometries of the surfaces of the fixed hub, corresponding thicknesses, or the like, may be inferred based on training data provided to the machine learning model.

Next, at block 714, and as previously described with respect to FIGS. 1A-4C, elongate member is formed (e.g., rendering data for an elongate member is generated) such that an elongate member extends from the needle support to the fixed hub. In other words, the elongate member rendering data is data defining a body that connects the needle support rendering data and the fixed hub rendering data. In some implementations, the elongate member rendering data is substantially linear. In other implementations, the elongate member rendering data includes at least one curved portion. In some implementations, the cross-sectional area of the elongate member rendering data may stay substantially the same from a location proximate the fixed hub rendering data to a location proximate the needle support rendering data. In other implementations, the cross-sectional area of the elongate member rendering data may stay constant or increase between a location proximate the fixed hub rendering data to a location proximate the needle support rendering data. In yet additional implementations, the cross-sectional area of the elongate member rendering data may stay constant or decrease between a location proximate the fixed hub rendering data to a location proximate the needle support rendering data. As previously described, in implementations where machine learning is implemented, the length of the elongate member, linearity or curvature of the elongate member, cross-sectional area geometry of the elongate member, rate of increase or decrease in cross-sectional area geometry of the elongate member, other geometries of the surfaces of the elongate member, or the like, may be inferred based on training data provided to the machine learning model.

Finally, at block 716, the dental guide may be fabricated. As previously described with respect to FIGS. 1A-4C, the rendering data may be combined between the fixed hub rendering data, the elongate member rendering data, and the needle support, each having been formed, and thus may be merged (if it hasn't already been merged by nature of the modeling process in the computer environment). This combined rendering data (i.e., the dental guide rendering data) may then be transformed into a STEP file, STL file, ICS file, or the like, and transmitted to a fabrication device such as an additive manufacturing device, CNC mill, or the like.

FIG. 8 illustrates a process flow of the administration of an anesthetic using a dental guide, in accordance with an implementation of the disclosure. Now that a fabricated dental guide has been obtained, the use of the dental guide as it pertains to the administration of anesthetic be described henceforth. At block 802, the fixed hub of the dental guide is applied to at least one tooth of the patient, typically at the bottom teeth (of the mandible). However, as previously described, other locations of affixing the fixed hub other than at least one tooth are contemplated. In some implementations, an adhesive may be used to secure the dental guide to the teeth or other anatomy of the patient. Such adhesives may include, but are not limited to, temporary dental cements and bonding agents, sealants, adhesive pastes, and glues.

At block 804, the operator inserts a needle of an injection device (e.g., a syringe or other injector) through the aperture of the needle support of the dental guide. The delivery tip (e.g., tip of a needle or injection device) may be in fluid communication with a syringe or other container having an anesthetic therein. In some implementations, the needle is connected to the injection device prior to inserting the delivery tip through the aperture. In other implementations, the needle is connected to the injection device after the injection device has been inserted through the aperture.

It shall be appreciated that the needle is connected to the injection device or syringe or other container via mechanisms including but not limited to a luer lock, slip tip, catheter tip, threaded tip, bayonet connection, screw-on connections, and so forth. Each of these mechanisms inherently provides for a portion of the needle-syringe assembly to flare outward at the proximal end of the needle, effectively creating a surface or body with at least one outer dimension larger than that of the aperture, preventing the needle from being inserted any further once the lingual surface of the needle support is reached by the connection mechanism.

Next, at block 806, the operator advances the injection device until the connection mechanism reaches the lingua surface of the needle support and is prevented from traveling further. As a result, the delivery tip of the injection device (the distal end) is at or near the target area (e.g., proximate the mandibular foramen of the patient). At block 808, the operator injects the anesthetic into the patient, such that the anesthetic is introduced at the target area. Once the predetermined amount of anesthetic has been injected, the process continues at block 810, where the operator removes the injection device (e.g., retracts the needle) through the aperture of the dental guide and away from the patient. Finally, at block 812, the dental guide may be removed from the at least one tooth of the patient.

Although many implementations of the present disclosure have just been described above, the present disclosure may be embodied in many different forms and should not be construed as limited to the implementations set forth herein; rather, these implementations are provided so that this disclosure will satisfy applicable legal requirements. Also, it will be understood that, where possible, any of the advantages, features, functions, devices, and/or operational aspects of any of the implementations of the present disclosure described and/or contemplated herein may be included in any of the other implementations of the present disclosure described and/or contemplated herein, and/or vice versa. In addition, where possible, any terms expressed in the singular form herein are meant to also include the plural form and/or vice versa, unless explicitly stated otherwise. Accordingly, the terms “a” and/or “an” shall mean “one or more,” even though the phrase “one or more” is also used herein. Like numbers refer to like elements throughout.

While certain exemplary implementations have been described and shown in the accompanying drawings, it is to be understood that such implementations are merely illustrative of, and not restrictive on, the broad disclosure, and that this disclosure not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described implementations can be configured without departing from the scope and spirit of the disclosure. Therefore, it is to be understood that, within the scope of the appended claims, the disclosure may be practiced other than as specifically described herein.

As will be appreciated by one of ordinary skill in the art in view of this disclosure, the present disclosure may include and/or be embodied as an apparatus (including, for example, a system, machine, device, computer program product, and/or the like), as a method (including, for example, a computer-implemented process, and/or the like), or as any combination of the foregoing. Accordingly, implementations of the present disclosure may take the form of an entirely apparatus implementation, an entirely software implementation (including firmware, resident software, micro-code, stored procedures in a database, or the like), an entirely hardware implementation, or an implementation combining the apparatus, software, and hardware aspects that may generally be referred to herein as a “system.” Furthermore, implementations of the present disclosure may take the form of a computer program product that includes a computer-readable storage medium having one or more computer-executable program code portions stored therein. As used herein, a processor, which may include one or more processors, may be “structured to” or “configured to” perform a certain function in a variety of ways, including, for example, by having one or more general-purpose circuits perform the function by executing one or more computer-executable program code portions embodied in a computer-readable medium, and/or by having one or more application-specific circuits perform the function.

It will be understood that any suitable computer-readable medium may be utilized. The computer-readable medium may include, but is not limited to, a non-transitory computer-readable medium, such as a tangible electronic, magnetic, optical, electromagnetic, infrared, and/or semiconductor system, device, and/or other apparatus. For example, in some implementations, the non-transitory computer-readable medium includes a tangible medium such as a portable computer diskette, a hard disk, a random access memory (RAM), a read-only memory (ROM), an erasable programmable read-only memory (EPROM or Flash memory), a compact disc read-only memory (CD-ROM), and/or some other tangible optical and/or magnetic storage device. In other implementations of the present disclosure, however, the computer-readable medium may be transitory, such as, for example, a propagation signal including computer-executable program code portions embodied therein.

One or more computer-executable program code portions (e.g., computer instructions) for carrying out operations of the present disclosure may include object-oriented, scripted, and/or unscripted programming languages, such as, for example, Java, Perl, Smalltalk, C++, SAS, SQL, Python, Objective C, JavaScript, and/or the like. In some implementations, the one or more computer-executable program code portions for carrying out operations of implementations of the present disclosure are written in conventional procedural programming languages, such as the “C” programming languages and/or similar programming languages. The computer program code may alternatively or additionally be written in one or more multi-paradigm programming languages, such as, for example, F #.

Some implementations of the present disclosure are described herein with reference to flowchart illustrations and/or block diagrams of apparatus and/or methods. It will be understood that each block included in the flowchart illustrations and/or block diagrams, and/or combinations of blocks included in the flowchart illustrations and/or block diagrams, may be implemented by one or more computer-executable program code portions. These one or more computer-executable program code portions may be provided to a processor of a general purpose computer, special purpose computer, and/or some other programmable data processing apparatus in order to produce a particular machine, such that the one or more computer-executable program code portions, which execute via the processor of the computer and/or other programmable data processing apparatus, create mechanisms for implementing the steps and/or functions represented by the flowchart(s) and/or block diagram block(s).

The one or more computer-executable program code portions may be stored in a transitory and/or non-transitory computer-readable medium (e.g. a memory) that can direct, instruct, and/or cause a computer and/or other programmable data processing apparatus to function in a particular manner, such that the computer-executable program code portions stored in the computer-readable medium produce an article of manufacture including instruction mechanisms which implement the steps and/or functions specified in the flowchart(s) and/or block diagram block(s).

The one or more computer-executable program code portions may also be loaded onto a computer, controller, and/or other programmable data processing apparatus to cause a series of operational steps to be performed on the computer and/or other programmable apparatus. In some implementations, this produces a computer-implemented process such that the one or more computer-executable program code portions which execute on the computer and/or other programmable apparatus provide operational steps to implement the steps specified in the flowchart(s) and/or the functions specified in the block diagram block(s). Alternatively, computer-implemented steps may be combined with, and/or replaced with, operator- and/or human-implemented steps in order to carry out an implementation of the present disclosure.

What is claimed is:

1. A dental guide comprising:
an elongate member defining a first end and a second end;
a needle support proximate the first end, the needle support defining an aperture structured to receive a needle; and
a fixed hub proximate the second end.

2. The dental guide of claim 1, wherein the fixed hub comprises a contoured portion structured to receive a portion of at least one tooth.

3. The dental guide of claim 2, wherein the fixed hub comprises a first material and the contoured portion comprises a second material.

4. The dental guide of claim 1, wherein the aperture comprises a bushing comprising an inner diameter sufficient to receive the needle.

5. The dental guide of claim 4, wherein the bushing comprises a metal.

6. The dental guide of claim 1, wherein at least one of the elongate member, the needle support, and the fixed hub, comprises an additive manufacturing material.

7. The dental guide of claim 6, wherein the additive manufacturing material comprises a polymer.

8. The dental guide of claim 6, wherein the additive manufacturing material comprises a metal.

9. The dental guide of claim 1, wherein the elongate member defines a longitudinal axis, and wherein the needle support extends from the elongate member in a direction substantially orthogonal to the longitudinal axis of the elongate member.

10. The dental guide of claim 1, wherein the needle support defines a lingual surface for receiving a proximal end of the needle.

11. The dental guide of claim 10, wherein, when installed on a patient having a mandibular foramen, the lingual surface is positioned between approximately 6 mm and approximately 50 mm from the mandibular foramen.

12. A method for injecting an anesthetic, comprising:
applying a dental guide to at least one tooth of a patient, wherein the dental guide comprises an aperture for providing access to a mandibular foramen;
receiving an injection device in the aperture, wherein the injection device is structured to deliver anesthetic;
advancing the injection device through the aperture until a delivery tip of the injection device is positioned proximate the mandibular foramen; and
injecting the anesthetic upon the delivery tip being proximate the mandibular foramen.

13. The method of claim 12 further comprising:
removing the injection device through the aperture of the dental guide; and
removing the dental guide from the at least one tooth of the patient.

14. The method of claim 12, wherein the injection device comprises a needle of a syringe.

15. The method of claim 12, wherein the dental guide comprises a fixed hub comprising a contoured portion, and wherein applying the dental guide to the at least one tooth of the patient comprises applying a temporary adhesive between the contoured portion and the at least one tooth.

16. A method of manufacturing a dental guide, the method comprising:
receiving image data of a patient, the image data comprising a mandibular foramen of the patient;
applying a linear marker in the image data to a position proximate the mandibular foramen;
determining an orientation of the linear marker based on anatomical features of the patient represented in the image data;
forming a needle support based on the orientation of the linear marker;
forming a fixed hub based on the anatomical features of the patient represented in the image data; and
forming an elongate member extending between the needle support and the fixed hub.

17. The method of claim 16, wherein the image data comprises first image data from an optical scan of the patient and second image data from a Computed Tomography (CT) scan of the patient.

18. The method of claim 16, further comprising:
arranging first image data of the patient and second image data of the patient to form combined image data;
wherein the image data of the patient comprises the combined image data.

19. The method of claim 16, wherein at least one of forming the needle support, forming the fixed hub, and forming the elongate member comprises using additive manufacturing.

20. The method of claim 16, wherein forming the needle support comprises forming an aperture in the needle support for receiving a needle.
